(19) United States
(12) Patent Application Publication
(10) Pub. No.: US 2023/0301805 A1
(43) Pub. Date: Sep. 28, 2023

(54) SLOPE ACCOMMODATING ORTHOTIC OR PROSTHETIC ANKLE JOINT OR BRACE AND ASSOCIATED METHODS

(71) Applicant: LOMA LINDA UNIVERSITY HEALTH, Loma Linda, CA (US)

(72) Inventors: Spencer Cutting, Loma Linda, CA (US); Michael Moor, Loma Linda, CA (US)

(73) Assignee: LOMA LINDA UNIVERSITY HEALTH, Loma Linda, CA (US)

(21) Appl. No.: 18/040,946

(22) PCT Filed: Aug. 6, 2021
(86) PCT No.: PCT/US2021/045029

§ 371 Date: Feb. 7, 2023

Related U.S. Application Data
(60) Provisional application No. 63/062,619, filed on Aug. 7, 2020.
(60) Provisional application No. 63/063,805, filed on Aug. 10, 2020.
(60) Provisional application No. 62/706,661, filed on Sep. 2, 2020.

(51) Int. Cl. A61F 2/66; A61F 2/70

(52) U.S. Cl. CPC A61F 2/6607; A61F 2/70; A61F 5/0111

(57) ABSTRACT

The present disclosure relates to an orthotic (or brace) and/or prosthetic ankle joint and associated methods adapted to enable adjustment of a talus section or ankle portion or brace with respect to a tibial section of the orthotic (or brace) and/or prosthetic ankle joint. The talus section or ankle portion of the orthotic (or brace) and/or prosthetic ankle joint is movably coupled to the tibial section with the alignment between the talus section and tibial section being adjustable between various positions by disengagement or engagement of a lock of an adjustment mechanism. As the lock is disengaged, the talus section is enabled to pivot with respect to the tibial section in at least one direction as the user moves along a slope.

### CROSS-REFERENCE TO RELATED APPLICATIONS

This application claims priority to, and the benefit of, U.S. Provisional Patent Application Ser No. 63/062,619, filed Aug. 7, 2020; U.S. Provisional Patent Application Ser No. 63/063,805, filed Aug. 10, 2020; and U.S. Provisional Patent Application Ser No. 62/706,661, Sep. 2, 2020. The entire contents of each of the above applications are hereby incorporated by reference.

### FIELD OF THE DISCLOSURE

The present disclosure generally relates to orthotic and prosthetic limbs. In particular, the present disclosure relates to an orthotic (brace) or prosthetic ankle joint and associated methods that includes adjusting and securing a foot/ankle portion of an orthotic (brace) or prosthetic ankle joint in varying alignments with respect to a tibial section of the orthotic (brace) or prosthetic ankle joint for accommodating movement along a slope.

### BACKGROUND OF THE DISCLOSURE

It is estimated that there are upwards of two million persons living in the United States who suffer from some type of debilitating injury or condition affecting their ability to walk freely. Prosthetic and orthotic devices have been used for quite some time as a replacement for amputated limbs, as well as to assist patients with paralysis or other conditions affecting use/motion of their lower extremities to assist with ambulation. Although improvements in orthotic and prosthetic devices have been made over the years, the problem with many orthotic and prosthetic devices has been an inability to substantially closely replicate a person's natural or normal (pre-loss) movement. For example, the human ankle serves as an important component for walking, navigating slopes, and even simpler tasks such as sitting, squatting and standing. In particular, ankle movement is important for providing the push-off or pre-swing phase of a person's gait, i.e., the initial force exerted when a person stands or starts to walk, as well as their balance during locomotion, particularly when moving over uneven ground or up a slope, ramp or stairs.

For patients with flaccid paralysis at the ankle, a brace (orthosis) may be provisioned to restore some functions and help allow ambulation. To prevent the user from collapsing during gait, and to provide propulsive force in the late phase of each step (i.e. “push-off”), the brace must prevent upward rotation (“dorsiflexion”) of the foot. The use of an articulated brace or prosthetic joint allows some motion, but often may hinder or limit the range of dorsiflexion, which limitation is termed a dorsiflexion stop or “dorsi-stop”. For example, where a user encounters an upward or downward slope, it may be necessary to change the angle of the foot and ankle with respect to the user's tibia. There are commercial options available to provide a dorsi-stop to an orthotic or prosthetic joint, but such devices often limit motion of the foot in relation to the proximal section of the tibia of the user, such that on upward slopes, the brace may allow insufficient dorsiflexion, which in turn creates excessive pressure on the proximal section of the device that may cause pain and knee hyperextension. On downward slopes, a plantar flexion stop may inhibit the foot and ankle to plantar-flex, causing the knee to flex, which is fatiguing and may inhibit or otherwise make situations risky for the user. These problems are amplified for a prosthetic user, who may experience substantial torque causing forceful knee flexion (which could cause substantial pain and potentially increase the chance of falling and being injured) when descending a slope. Without a dorsiflexion stop there is nothing to contribute to knee extension which too can be fatiguing.

Accordingly, it may be seen that a need exists for an orthotic or prosthetic ankle joint that is enabled to adjust alignment of the foot or ankle brace portion with respect to the tibial portion of the orthotic or prosthesis, and which addresses the foregoing and other related and unrelated problems in the art.

### SUMMARY OF THE DISCLOSURE

Briefly described, the present disclosure generally is directed to an orthotic ankle brace for patients with flaccid paralysis or other types of paralysis affecting their lower leg and ankle to assist in control of the patient's foot and ankle, and/or to a prosthetic ankle joint for use as a replacement ankle and/or lower leg in bilateral amputations. In one aspect, the present disclosure is directed to an anatomically aligned ankle joint orthotic or prosthetic. The orthotic (or brace) and/or prosthetic ankle joint may include a tibial section and a talus or ankle portion, and may include a shell or sleeve linked to the tibial section and configured to fit about the user's shin, such as for application as an orthotic brace or support. An adjustment mechanism will be coupled to the tibial section and engages and disengages with/from the talus or ankle portion to enable pivoting/dorsiflexion movement or other adjustment of the talus or ankle. The talus and tibial section further may be movably/pivotally coupled or connected together by a connector such as a rod or pin.

In addition, a biasing element such as a spring, can be provided between the talus and the adjustment mechanism or a portion of the tibial section to provide a dorsiflexion torque or biasing force for directing or urging the foot of the user toward a substantially upward orientation to assist in dorsiflexion and lifting of the user's foot during the swing phase of the user's gait and help slow descent of their foot after heel strike. Still further, in some embodiments, such as for prosthetic replacement of a user's lower leg or foot/ankle, a prosthetic foot also may be coupled to the talus.

In one aspect, the adjustment mechanism of the orthotic (or brace) and/or prosthetic ankle joint will be configured to lock and unlock the talus from its position/alignment with respect to the tibial section to enable re-alignment of the ankle joint based on the orientation of the proximal (tibial/calf) section of the orthotic and/or prosthetic ankle to a generally vertical axis. Such adjustment may be made without necessarily having to accommodate the orientation of the foot section to the tibial section.

In some embodiments, the adjustment mechanism may include a pendulum that may be engaged and/or moved to cause engagement/disengagement of a lock of the adjustment mechanism to enable realignment of the talus or ankle portion. The pendulum may be coupled to the lock and to a weight or an actuator/drive that will cause movement of the pendulum as the user moves along a sloped surface or terrain. Movement of the pendulum will cause the lock to engage with and/or disengage from the talus of the ankle joint to secure the ankle joint in a substantially fixed orientation during ambulation of the user, or for enabling dorsiflexion of the talus to adjust and realign with respect to the tibial section as needed, such as during walking and further, when the user encounters and/or moves up or down a slope.

The adjustment mechanism also may be configured to be operable substantially automatically. For example, as the user encounters and begins movement along a slope, the pendulum of the adjustment mechanism of the ankle joint may be mechanically driven by a weight, by a spring or other biasing member, a levering mechanism; or may be electronically driven by an actuator such as a solenoid, linear clutch or motor, causing the pendulum to move for engaging and disengaging the lock. In response to the disengagement of the lock, the talus or ankle portion is enabled to move with respect to the tibial section, allowing the talus or ankle to realign substantially automatically or in a manner that may generally mimics the natural movement/dorsiflexion motion of the user's ankle to accommodate movement of the user, including movement and/or standing of the user along an upward or downward slope.

In alternative embodiments, the adjustment mechanism may be driven with other types of actuation mechanisms. For example, a pendulum, rack and pinion or camming system may be engaged/disengaged and moved by a mechanical or electromechanical drive. In addition, a sensor such as a tilt sensor, gyroscope, accelerometer, or other, orientation or motion sensor/detector may be provided for sensing/detecting a change in orientation or direction of the talus with respect the tibial section, and in response to such detection, an actuator may be engaged to lock and unlock the adjustment mechanism of the ankle joint.

In another embodiment of the present disclosure, an orthotic or prosthetic ankle joint includes a tibial section, a talus section, and an adjustment mechanism. The talus section is movably connected to the tibial section. The adjustment mechanism is configured to selectively prevent additional dorsiflexion of the talus section relative to the tibial section to accommodate movement of a user.

In embodiments, the adjustment mechanism includes a lock and a pendulum. The pendulum may be adapted to move in response to movement of the user such that the lock is locked to prevent additional dorsiflexion of the talus section with respect to the tibial section and unlocks to allow realignment of the talus section with respect to the tibial section. When locked, the lock may fix the talus section with respect to the tibial section. The joint may include a foot coupled to the talus section.

In some embodiments, the talus section includes a series of teeth that are engageable by the lock. Engagement of the lock with a tooth of the series of teeth may prevent additional dorsiflexion of the talus section with respect to the tibial section. The lock may be pivotally mounted to the tibial section. The lock may comprise an engaging end for engaging the series of teeth to prevent additional dorsiflexion of the talus section with respect to the tibial section. The lock may be operably coupled to the pendulum such that the pendulum pivots the engaging end into and out of engagement with the series of teeth.

In certain embodiments, the lock is slidably mounted to the tibial section. The lock may include an engaging end for engaging the series of teeth to prevent additional dorsiflexion of the talus section with respect to the tibial section. The lock may include a rack and the pendulum may be operably coupled to a pinion engaged with the rack. The pendulum may pivot the pinion to translate the lock into and out of engagement with the series of teeth.

In particular embodiments, the tibial section is pivotably connected to the talus section by a connector. The adjustment mechanism may include a pendulum pivotably disposed about the connector. The tibial section may include a series of teeth. The lock may be operably coupled to the pendulum such that the pendulum moves the lock into and out of engagement with the series of teeth to prevent additional dorsiflexion of the talus section with respect to the tibial section. The talus section may include a protecting portion that includes the series of teeth. The projecting portion may define a cavity with the lock disposed within the cavity of the projection portion.

In some embodiments, the adjustment mechanism includes a lock connected to one or more of the talus section or tibial section. The actuator may be for engaging the lock to prevent additional dorsiflexion of the talus section with respect to the tibial section and disengaging the lock to enable movement of the talus section with respect to the tibial section. The actuator may comprise a mechanical actuator or an electromechanical actuator.

In certain embodiments, the adjustment mechanism includes a position sensor and an electromechanical actuator. The position sensor may be configured to sense a change in orientation or direction of movement of the talus with respect to the tibial section. The electromechanical actuator may be configured to selectively allow movement of the talus section with respect to the tibial section in response to the orientation or the direction of movement of the talus section with respect to the tibial section. The adjustment mechanism may include a locking pin that is translatable between a first position and a second position. In the first position, movement of the talus section with respect to the tibial section may be allowed. In the second position, additional dorsiflexion of the talus section with respect to the tibial section may be prevented. The electromechanical actuator may be operably coupled to the locking pin to translate the locking pin between the first position and the second position. The adjustment mechanism may include a clutch that couples the electromechanical actuator to the locking pin.

In some embodiments, the joint includes a biasing member connected to the talus section and the tibial section. The biasing member may be configured to cause or aid dorsiflexion of the talus section with respect to the tibial section.

In another embodiment of the present disclosure, the method to operate an orthotic and prosthetic ankle joint for accommodating movement of a user along a slope includes mounting an orthotic and prosthetic ankle joint to an ankle region of a user, moving an engaging end of a lock into engagement with teeth of the talus section when an angle of a user's shin moves forwardly in a user selected angle, locking the tibial section and talus section at the user selected angle, enabling push off as part of the user's normal gait during a walking motion when raising a heel of a foot of the user onto which the joint is mounted, and reengaging the talus section with the lock to secure the talus section and the tibial section together for accommodating movement of a user along a slope. The orthotic and prosthetic ankle joint including a tibial section, a talus section movably connected to the tibial section, and adjustment mechanism that is configured to adjust an alignment of the talus section with respect to the tibial section. The adjustment mechanism may include the lock.

In some embodiments, the method includes locking and unlocking the lock with a pendulum in response to the user moving along a slope to enable movement and realignment of the talus section with respect to the tibial section. The method may include engaging and disengaging the lock with an actuator to enable movement of the talus section with respect to the tibial section. Engaging and disengaging the lock includes the actuator may include a mechanical actuator or an electromechanical actuator.

Various objects, features and advantages of the present disclosure will become apparent to those skilled in the art upon a review of the following detailed description, when taken in conjunction with the accompanying drawings. Further, to the extent consistent, any of the embodiments or aspects described herein may be used in conjunction with any or all of the other embodiments or aspects described herein.

### BRIEF DESCRIPTION OF THE DRAWINGS

FIG. 1A is a side view of an embodiment of an orthotic (or brace) and/or prosthetic ankle joint according to the principles of the present disclosure.

FIGS. 1B-1C are perspective views of the orthotic (or brace) and/or prosthetic ankle joint of FIG. 1A according to the principles of the present disclosure.

FIG. 1D is a perspective view of the orthotic (or brace) and/or prosthetic ankle joint of FIG. 1A according to the principles of the present disclosure, illustrating an embodiment of an adjustable linkage between the lock and pendulum.

FIG. 1E is a perspective view of the orthotic (or brace) and/or prosthetic ankle joint of FIG. 1A according to the principles of the present disclosure, illustrating another embodiment of the pendulum.

FIG. 1F illustrates a walking motion of a user utilizing the orthotic (or brace) and/or prosthetic ankle joint of FIGS. 1A-1C according to the principles of the present disclosure.

FIG. 2 is a side view of another embodiment of an orthotic (or brace) and/or prosthetic ankle joint according to the principles of the present disclosure.

FIG. 3 is a side view illustrating an additional embodiment of an orthotic (or brace) and/or prosthetic ankle joint according to the principles of the present disclosure.

FIG. 4 is a side view of still a further embodiment of an orthotic (or brace) and/or prosthetic ankle joint according to the principles of the present disclosure, with a prosthetic foot attached to a lower portion of the talus of the orthotic (brace) and/or prosthetic ankle joint.

Those skilled in the art will appreciate and understand that, according to common practice, the various features of the drawings discussed below are not necessarily drawn to scale, and that the dimensions of various features and elements of the drawings may be expanded or reduced to more clearly illustrate the embodiments of the present disclosure as described herein.

### DETAILED DESCRIPTION OF THE DISCLOSURE

The present disclosure will now be described more fully hereinafter with reference to example embodiments thereof with reference to the drawings in which like reference numerals designate identical or corresponding elements in each of the several views. These example embodiments are described so that this disclosure will be thorough and complete, and will fully convey the scope of the disclosure to those skilled in the art. Features from one embodiment or aspect can be combined with features from any other embodiment or aspect in any appropriate combination. For example, any individual or collective features of method aspects or embodiments can be applied to apparatus, product, or component aspects or embodiments and vice versa. The disclosure may be embodied in many different forms and should not be construed as limited to the embodiments set forth herein; rather, these embodiments are provided so that this disclosure will satisfy applicable legal requirements. As used in the specification and the appended claims, the singular forms “a,” “an,” “the,” and the like include plural referents unless the context clearly dictates otherwise. In addition, while reference may be made herein to quantitative measures, values, geometric relationships or the like, unless otherwise stated, any one or more if not all of these may be absolute or approximate to account for acceptable variations that may occur, such as those due to manufacturing or engineering tolerances or the like.

As used herein, the term “proximal” refers to the portion of the device or component thereof that is closer to a torso of a patient or user and the term “distal” refers to the portion of the device or component thereof that is farther from the torso of a patient or user.

This disclosure relates generally to a prosthetic ankle joint for use as a replacement limb for patients or users that have had amputations necessitating replacement of a lower leg portion of such a patient or user, and/or as a brace or orthotic assist for patients who are experiencing paralysis or other debilitating condition affecting the use of a lower extremity (e.g. a foot or ankle) affecting their ability to walk.

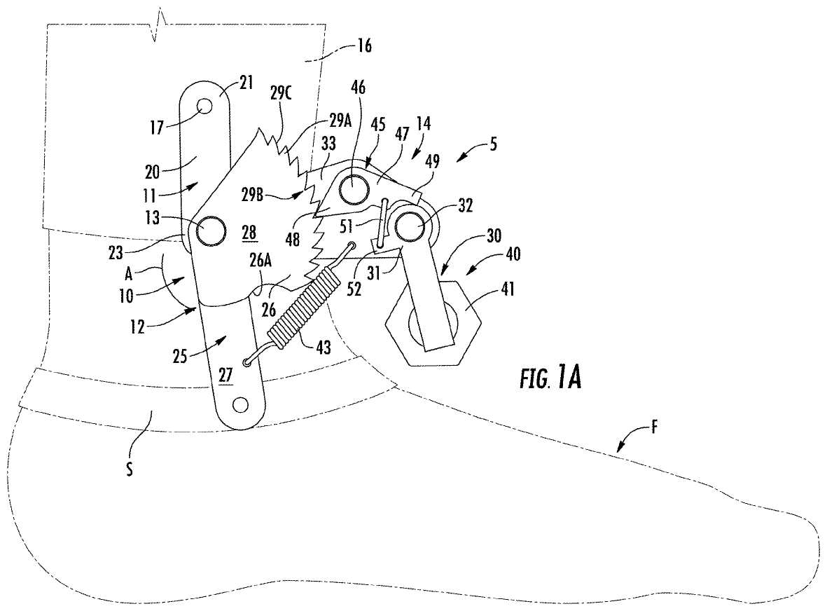

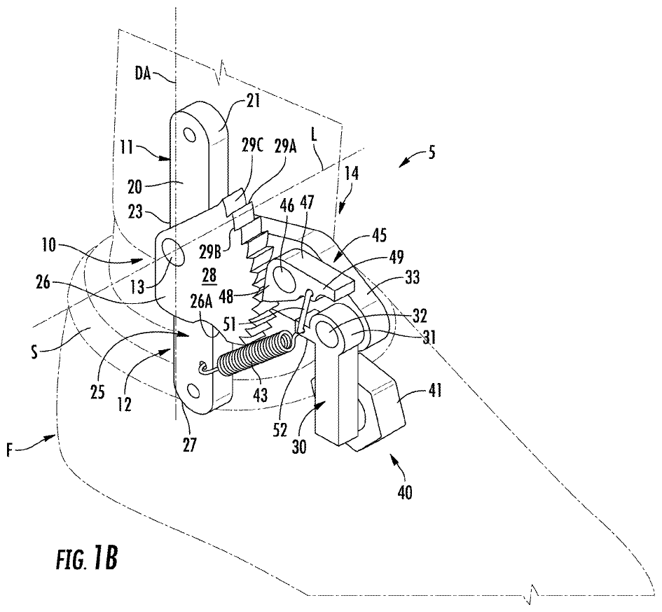

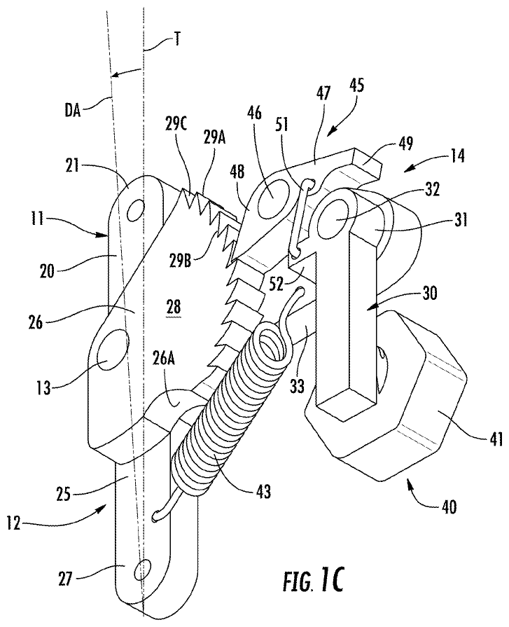

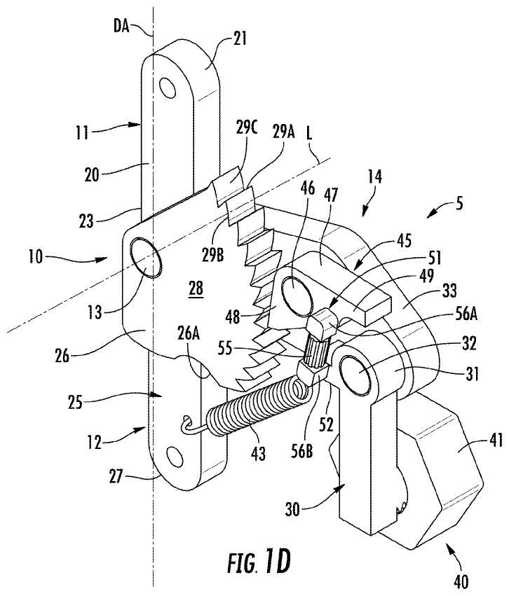

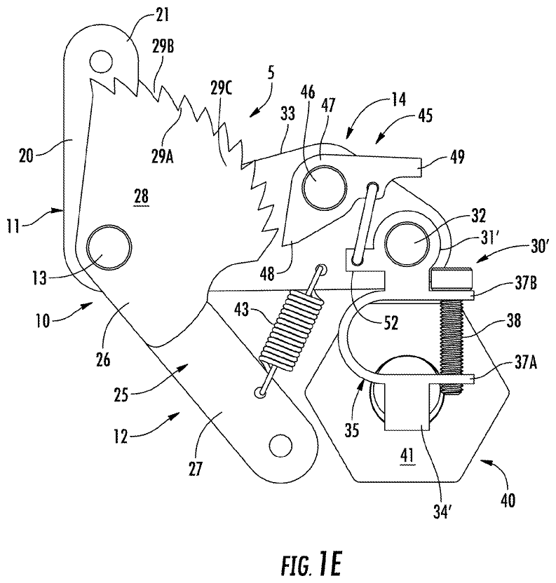

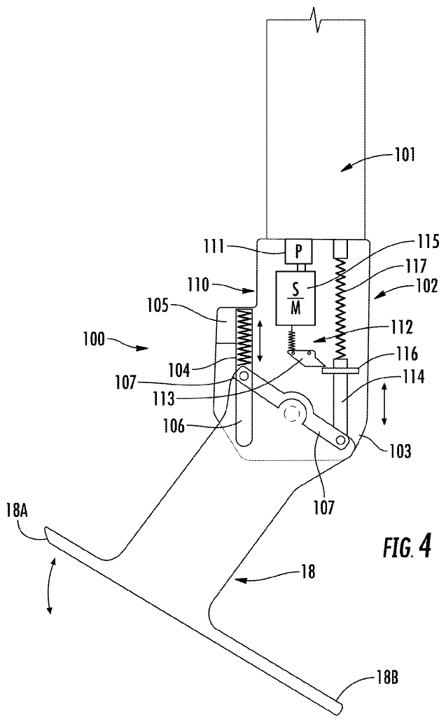

As illustrated in FIGS. 1A-1F in an exemplary embodiment, an orthotic (or brace) and/or prosthetic ankle joint 5 includes a body 10, having a tibial section 11, a talus section or ankle portion 12, a connector 13, such as a pin or rod that pivotally connects or couples the tibial section to the talus section or ankle portion, and an adjustment mechanism 14. As indicated in FIGS. 1A-C, for some applications or embodiments where the orthotic (or brace) and/or prosthetic ankle joint 5 is generally to be used as an orthotic brace, support or assist for a user's existing ankle and foot (shown at A/F in phantom lines in FIG. 1A), as will be understood by those skilled in the art, a sleeve, shell or other attachment member 16 may be coupled to the tibial section 11, such as by fasteners 17. The sleeve 16 may be shaped or otherwise configured to fit about the user's shin, and may be secured to the user's shin, i.e., with a strap S or other, similar connector, extended about the user's shin to attach the orthotic and/or prosthetic ankle joint 5 to the user's leg, as will be understood by those skilled in the art. In addition, in some applications/embodiments, such as where the orthotic (brace) and/or prosthetic ankle joint 5 is to be used as a prosthetic limb replacement for replacement of a user's lower leg or ankle, a foot 18, such as indicated in FIG. 4, also may be coupled to a lower end or section of the talus.

The talus and tibial sections of the orthotic and/or prosthetic ankle joint 5 generally will be formed from durable, medical grade materials such as plastics, composites, aluminum, steel and other, similar lightweight materials as will be understood by those skilled in the art.

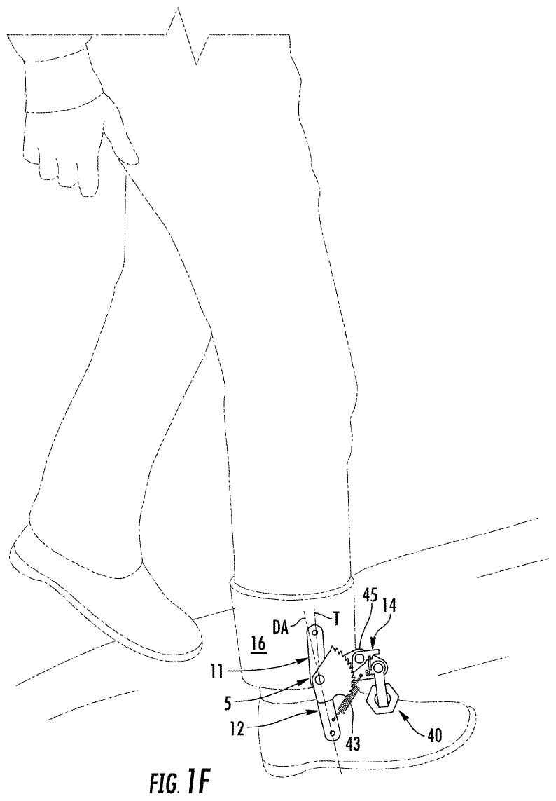

The tibial section 11, as illustrated in FIGS. 1A-2, generally will include an elongated body 20 that may be sized and/or otherwise configured to match the user's physiology. For example, the tibial section may include a rod or plate or other member of a length approximately equivalent to the user's sound or existing tibia, or at least a portion thereof. In some embodiments, the tibial section further will have a configuration and/or structure designed to support the user's weight. The body 20 of the tibial section 11 will have an upper or proximal end 21 that may attach to the shell or sleeve 16 (FIG. 1A), or to an existing portion of the user's tibia, or, in some embodiments for some applications such as a prosthetic replacement limb, to a connector for attachment to the user's knee joint; and a lower or distal end 23. The lower or distal end 23 of the tibial section 11 generally will be pivotally attached to an upper end 26 of the body 25 of the talus section or ankle portion 12 of the orthotic (or brace) and/or prosthetic ankle joint 5 by the connector 13. Other, alternative constructions, including the use of pivoting connectors, also may be provided for attachment/coupling of/connecting the tibial section to the talus section or ankle portion.

As further illustrated in FIGS. 1A-2, the talus section or ankle portion 12, in some embodiments, generally will include an elongated body 25 with an upper portion 26 that is pivotally coupled to the lower end 23 of the body 20 of the tibial section in an arrangement that enables rotation or pivoting movement of the talus with respect to the tibial section and in at least one direction along a substantially laterally oriented, transverse joint axis L (FIGS. 1B and 1D). In some embodiments, such as for use as a prosthetic limb replacement, the lower portion 27 of the talus further may include a connector or linkage adapted to pivotally couple the talus to a prosthetic foot or lower brace portion (FIG. 4). In addition, depending on the physiology of the user, the talus 12 (FIGS. 1A-1E) further may be oriented at a slight angle or cant along a coronal joint axis with respect to the tibial section 11 as will be understood by those skilled in the art.

In some embodiments, the adjustment mechanism 14 may include a pendulum 30 pivotally mounted at a proximal or upper end 31 thereof, such as by a pin or other connection 32 to an adjustment support plate 33 or other, similar support, and a distal or lower end 34. The adjustment plate 33 generally will be formed with or attached to the body 20 of the tibial section 11, adjacent the lower end thereof. The adjustment plate may have a varying configuration as needed to fit the physiology of the wearer and/or the application, i.e., depending on whether the orthotic and/or prosthetic ankle joint 5 is being used as an orthotic brace or as a prosthetic replacement, and further may be affixed to or substantially integrally formed with the tibial section.

As indicated in FIGS. 1A-2, the pendulum may have an elongated body 35 that may be substantially rectangular, square, cubical, or may have varying other configurations, and will project downwardly from the pivotal attachment of its upper end 31 at the support plate, to the distal or lower end 34 below a lower edge of the support plate. The lower end 34 of the pendulum further generally may be attached to an actuator 40, which may include a mechanical actuation on device such as a weight 41, or an electromechanical actuator or drive, or other, similar device, adapted to cause movement of the pendulum, for example, causing a swinging or other movement of the lower end of the pendulum toward or away from the talus, as a user encounters an upward or downward slope. The operation of the pendulum further is generally independent of what the foot (talus) section is doing or how it is moving, with the pendulum generally swinging or moving with respect to a substantially vertical axis.

The pendulum further can have varying configurations. For example, FIG. 1E illustrates another embodiment of a pendulum 30′, wherein the body 35′ of the pendulum is adjustable in length so as to enable adjustment of the positioning of the actuator/weight 40/41. The body 35′ of the pendulum 30′ can have an upper end 31′ pivotally connected to the support plate 33, a lower end 34′ coupled to the actuator/weight, and a flexible or compressible intermediate body section 36. In one embodiment, the intermediate body section can have a generally U or C-shaped configuration, with spaced arms or flanges 37A/37B connected at their free ends by a fastener 38. The fastener 38 can include a set-screw or other type of adjustment mechanism that engages and draws the arms or flanges 37A/37B together or urges the arms apart to enable adjustment of the length of the pendulum as needed thereby adjusting the position of the pendulum in relation to vertical. This enables fine tuning of the point where the dorsiflexion stop engages the tibial section for push off.

In one embodiment, as illustrated in FIGS. 1A-1C, the upper end 26 of the body of the talus section or ankle portion 12 may be formed with a substantially arcuate or enlarged section or distal projecting portion 28 having a series of teeth or projections 29A formed along a substantially arcuate peripheral surface or edge 29B thereof, with the teeth or projections 29A defining a series or recesses or gaps 29C there between. The number of teeth 29A may be varied to enable closer or finer adjustments of a substantially vertically extending angle of the talus with respect to the tibial section. The upper portion 26 of the body of the talus section or ankle portion also may be configured so as to overlap the lower portion 27 of the body of the talus, with a lower edge of the upper portion 26 of the talus section slightly outwardly from the lower portion of the talus, and with the lower portion 27 of the talus spaced below the lower end of the tibial section to define a gap, inset or area of separation, indicated at 26A.

The gap or inset 26A defined along the body 25 of the talus section or ankle portion 12, may be configured and/or sized to receive a malleolus of a user's existing ankle, which can help provide a more substantially fitted engagement of the orthotic (or brace) and/or prosthetic ankle joint 5 to the user's ankle. For example, as indicated in FIG. 1A, the embodiments or aspects where the orthotic and/or prosthetic ankle joint 5 is used as an orthotic brace to assist patients with a paralysis along their ankle and foot, the tibial section may be coupled to the shell or sleeve 16, which will extend along the shin portion of the user. The user's existing ankle joint or malleolus, on either the inside or outside of the user's leg, may be received and fit or rest within the inset or area of separation 26A between the tibial section and the talus section or ankle portion 12.

The lower portion 27 or the talus 12 further may be coupled to a shoe or to a plate or other attachment mechanism, or about the lower leg/ankle of the user such as by adjustable straps S or other connectors inserted through the lower portion of the talus or attached to the one or more openings 27A, as indicated in FIGS. 1A-1B, to secure the orthotic (or brace) and/or prosthetic ankle joint 5 about the lower leg and ankle of the user.

In addition, the orthotic (or brace) and/or prosthetic ankle joint 5 and/or the individual tibial section and talus section or ankle portion thereof may be formed in varying sizes and configurations to enable custom fitting of the orthotic and/or prosthetic ankle joint to a particular user. Still further, the orthotic (or brace) and/or prosthetic ankle joint also may be formed in a set or prescribed series of sizes, e.g. pediatric, young adult, adult, and/or larger sizes configured to fit individuals up to approximately 300 pounds. Other sizing options also may be used.

As further indicated in FIGS. 1A-1E, the adjustment mechanism may include a lock 45 pivotally mounted to the adjustment plate 33 attached or formed as a part of the tibial section, such as by a pin or hinged connector 46. The lock 45 generally may include a body 47 having a pointed or hooked, proximal or engaging end or portion 48 and a distal or rear end 49. A linkage, ring or other, similar connector 51 may be attached to the body of the lock at or adjacent to the rear end 49 thereof, and further will be attached to the pendulum 30, for example, being attached to a flange 52 or similar connection feature formed adjacent the upper end 31 of the pendulum. As a result, as indicated in FIGS. 1A and 1B, as the pendulum moves, e.g., as the lower end of the pendulum pivots or swings with respect to the support plate, the linkage or connector between the body 47 of the lock 45 and the upper end of the pendulum causes the lock to be urged or pivoted upwardly or downwardly, moving the proximal or engaging end 48 thereof out of engagement with the teeth 29A of the talus section or ankle portion.

For example, as generally illustrated in FIG. 1F, during a walking movement, and/or as the user encounters a sloped surface/terrain and walks there along, the angle T of their tibia or shin with respect to their talus changes as the tibia of the user tilts and moves forwardly to push off, and rearward when returning to a resting position and/or when taking a step with the opposite leg/foot. As the user's shin tilts forwardly to an appropriate angle T with respect to an angle of dorsiflexion DA, as indicated in FIG. 1C, the proximal or engaging end 48 of the lock 45 may be moved into engagement with the teeth of the talus section or ankle portion of the orthotic and/or prosthetic ankle joint or brace, locking the tibial and talus sections at an appropriate angle, and forcing the heel of the user to raise for enabling push off as part of the user's normal gait or walking motion. In some embodiments, engagement of the locking end 48 of the lock 45 may prevent additional dorsiflexion of the talus section relative to the tibial section. Once the user stops walking or moving forwardly, such as when they take a step with their other foot or leg, as their shin tilts back toward a more natural alignment/orientation with respect to the user's talus, the lock may disengage from the teeth of the talus section or ankle portion, with the user's foot and heel urged to a generally flat lying orientation for supporting the user in either a standing position or as they take a step with their opposite foot.

In addition, the angle at which the pointed or hooked end 48 of the lock 45 engages with the teeth 29A of the body 26 of the talus can be configured to be adjustable in order to account for variations in the angle at which the joint is attached to the talus and tibial sections of the orthotic (or brace) and/or prosthetic ankle joint 5, and for the unique anatomy of the user. For example, due to a lack of consensus by technicians or orthotists/prosthetists fitting the orthotic (or brace) and/or prosthetic ankle joint in the field, as well as sometimes wide differences in opinions/beliefs of individual orthotists/prosthetists as to the optimal angle at which locking of the orthotic (or brace) and/or prosthetic ankle joint should occur, some adjustments or variations of the length of the connector or linkage 51 may be necessary.

In addition, the linkage or connector 51 also can be configured to be adjustable as needed. For example, as illustrated in FIG. 1D, in some embodiments, the linkage 51 can include a turnbuckle 55 or other adjustment mechanism or device that can enable the length of the linkage or connector 51 to be extended or retracted to provide for adjustments in the field. As the body of the turnbuckle 55 is rotated, the ends 56A/56B of the linkage 51, which are coupled to the body of the lock 45 and to the flange 52 of the pendulum 30, will be drawn toward each other, or urged apart, providing adjustment of the length of the linkage 51 without requiring a replacement of the linkage or other component parts of the orthotic (or brace) and/or prosthetic ankle joint. In other embodiments, the angle between the tibial section 20 and the support plate 33 could be altered or configured to be adjustable, and/or the orientation of the flange 52 of the pendulum 30 (to which the linkage or connector 51 is attached) also can be configured to be adjustable or variable.

In some embodiments, as indicated in FIGS. 1A-2, the actuator 40 may include a weight 41, or other, similar mechanical actuator. The weight 41 will exert a downward pulling force on the pendulum, such as indicated by arrow 42, which downward pulling force will generally cause the pendulum 30 to swing or move in a self-centering movement to a substantially vertically oriented or dead-center position. The lock 45 is initially disengaged such that the talus is enabled and allowed to move, swing, or pivot along a substantially laterally or transversely aligned joint axis extending through the connector 13. Thus, the talus section or ankle portion or brace may adjust or change its alignment with respect to the tibial section and/or with respect to a substantially vertical dorsiflexion axis DA (as indicated in FIG. 1D) extending along the tibial section to accommodate a change in direction of the talus section or ankle portion with respect to the tibial section as the user encounters and moves along an upward or downward slope. Such adjustment generally may be made without having to adjust for or otherwise accommodate an orientation of the user's foot with respect to the tibial section.

A spring 43 or other biasing mechanism can be provided between the support plate 33 and the lower section or portion 27 of the talus 12, as indicated in FIG. 1A. This spring will tend to urge or draw the lower end of the talus toward the support plate, which can provide a dorsiflexion torque or biasing force to help lift the user's foot during the swing phase of their gait/walking motion, and which can help slow the descent of the user's foot after heel strike. The spring 43 can be added to help account for dorsiflexion weakness/paralysis experienced by a user of the orthotic and/or prosthetic ankle joint of brace 5, whereas the central mechanism of the orthotic and/or prosthetic ankle joint of brace 5 provided by the engagement between the teeth of the talus upper section and the lock 45 can help adjust and/or stop undue dorsiflexion during the stance phase of gait to compensate for plantar flexion weakness/paralysis.

As the user encounters a sloped surface or terrain, the angle of their talus with respect to their tibia changes, causing the talus to shift, swing or otherwise be reoriented with respect to the talus. During such motion, the downward pulling force exerted by the weight on the pendulum toward a dead-centered position such that, as the angle T of the user's tibia with respect to their talus reaches an angle necessary for push-off, the lock body will be pivoted sufficient to engage the teeth 29A of the upper portion of the talus, generally securing the talus in a new, adjusted alignment with respect to the tibial section, which accommodates for the slope or uneven terrain, and enables push-off of the user's foot from a terminal stance position. Thereafter, the lock may disengage the talus with respect to the tibial section to enable a more natural movement of the talus as the user continues walking.

In an alternative embodiment, shown in FIG. 2, the lock of the adjustment mechanism 14 may comprise or include a rack and pinion mechanism 60. In this embodiment, the upper end of the pendulum 30 may be formed with a series of teeth 61 that are configured to engage the teeth 62 of a corresponding rack portion 63 that forms the body of a lock 65, and which can be slidably supported along the support or adjustment plate 33 by a guide or bearing slide 66. As the pendulum is swung or rotated, such as by the self-centering movement of the weight as the user moves along an upward or downward slope, the teeth of the pendulum may disengage and engage the rack teeth of the lock, enabling the rack/body of the lock to move or translate toward and away from the talus. The rack further will include recesses 64A defined between a series of teeth 64B configured or adapted to engage the teeth of the talus for locking the talus in varying alignments with respect to the tibial section. As noted, the number of teeth along the rack 63 further may be varied to provide a greater or finer variation of the angle of orientation of the talus with respect to the tibial section.

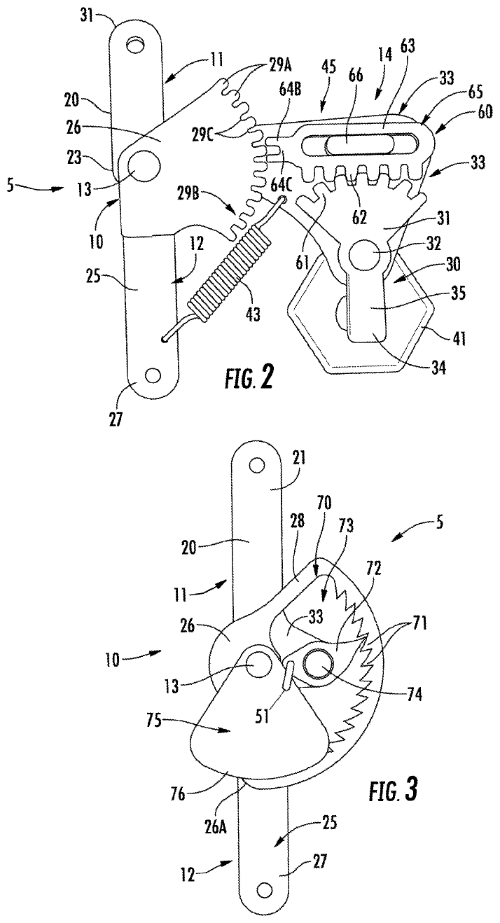

In addition, in still further embodiments, the adjustment mechanism 14 may comprise a camming mechanism 70. For example, in one embodiment as illustrated in FIG. 3, a camming mechanism 70 can be used in place of the locks such as shown in FIGS. 1A-2, and can include a series of teeth 71 or other camming surfaces that are engaged by a cam or pivoting lock member 72 attached to the support plate 33 formed with or attached to the tibial section 11. In addition, in some embodiments such as illustrated in FIG. 3, the adjustment mechanism 14 may be mounted internally within a cavity 73 defined along the upper portion 26 of the body 25 of the talus section or ankle portion 12, which may provide a smaller or reduced profile, or more compact construction for the orthotic and/or prosthetic ankle joint or brace.

As illustrated in FIG. 3, the pendulum or other mechanical actuator 75 generally is shown connected to an opposite end of cam 72. The pendulum also may be coupled to a weight and/or have a construction similar to the embodiments/configurations of the pendulums as generally illustrated in FIGS. 1A-2. Alternatively, in lieu of a separate weight attached to the lower end 76 of the pendulum, the pendulum itself may be constructed of denser or heavier materials, or the lower end 76 of the pendulum 75 may be loaded with a denser or heavier material to eliminate the use of a weight, and allow the lower end of the pendulum itself to act as the weight or mechanical actuator.

As the user walks forwardly, causing their shin to tilt and move toward a more forward angle with respect to the talus section or ankle portion, the talus section or ankle portion will substantially automatically shift or otherwise move or adjust its dorsiflexion angle of the user's talus with respect to their tibia, particularly as the user is moving along an upward or downward slope. As the angle between the user's talus and tibia reaches an appropriate angle for push-off, the cam portion of the lock will engage with the cam surfaces formed along the inside of the talus section or ankle portion, locking the talus and tibia so as to force the heel of the user off the ground and enabling push-off of the user's foot. The dorsiflexion angle of the tibial section with respect to the talus section or ankle portion thus is not restricted to a limited range or movement, but rather enabled to pivot or be substantially reoriented across a wider range of movements that can more closely match the user's natural gait or walking motion.

In various embodiments, other mechanical and/or electro-mechanical actuators may be coupled to the pendulum for directing and/or controlling movement thereof, or may be coupled to the lock of the adjustment mechanism without a pendulum. For example, in place of a weight as illustrated in the figures, a pneumatic or hydraulic cylinder, motor, linear clutch or other similar actuator or drive may be used for locking and unlocking the talus and tibial sections in a designed alignment.

Still further, in embodiments such as where an electromechanical actuator or drive mechanism, such as a solenoid, cylinder, motor, or linear clutch, or other electronic drive is used, e.g. as indicated in FIG. 4, the orthotic (or brace) and/or prosthetic ankle joint 5 also may include one or more sensors, such as a tilt sensor, gyroscope, or accelerometer linked to the actuator. The sensors will be configured to detect a change in position or orientation of the user's tibia, i.e., as the user starts to walk up or down a sloped surface or uneven terrain, the change in direction or orientation may be detected by a sensor, which communicates with the actuator, causing the actuator to disengage the lock of the adjustment mechanism and enable the talus section or ankle portion of the orthotic (or brace) and/or prosthetic ankle joint to substantially automatically readjust its position or alignment with respect to the tibial section.

The orthotic and/or prosthetic ankle joint further may be applied to or incorporated as part of a prosthetic foot and ankle. Still further, by creating a locking joint with a construction robust enough to bear a user's weight, and with standard adaptors above and below the ankle joint to enable attachment in series with standard prosthetic components, the orthotic or prosthetic ankle joint of the present disclosure may be integrated into a variety of foot/ankle prosthesis.

By way of example, and as noted, the orthotic (brace) and/or prosthetic ankle joint may be configured for use as either an orthotic (brace), and/or also can be used as a prosthetic or replacement limb. For example, in the embodiment illustrated in FIG. 4, an orthotic (brace) or prosthetic ankle joint 100 may have a tibial section 101 coupled to a talus section or ankle portion 102, and with a prosthetic foot 18 pivotally mounted to the lower end or portion 103 of the talus 102. An ankle dorsiflexion spring 104 may be mounted along an internal cavity 106 within the body 105 of the talus 102, and generally will provide a biasing force directed against an upward edge or portion 107 of the prosthetic foot 18, generally urging the heel section 18A of the prosthetic foot 18 toward a downward, or substantially flat lowered alignment. The upper end 108 of the body of the talus generally will be attached to the tibial section 101, which may include a rod or other, similar prosthetic body, such as by fasteners or other connection.

As further illustrated in FIG. 4, an adjustment mechanism 110 that includes an electro-mechanical actuator 115, such as a solenoid, pneumatic or hydraulic cylinder, or a motor can be mounted within the body of the talus 102. The electromechanical actuator also may include or be coupled to a rechargeable battery or other, similar power source that may be located within or along the talus and/or the tibial section. In some embodiments, the power source may be remote to the orthotic (brace) or prosthetic ankle joint 100. A position sensor 111, such as orientation or acceleration sensor, etc., may be coupled to the electromechanical actuator 115 and may be configured to sense changes in the position of the tibial section with respect to the talus as the angle between the tibial section and talus section changes during movement or locomotion of the user. In response, the electromechanical actuator can engage a clutch type lock mechanism 112 to release or secure a pivoting lock member 113 for enabling movement of a locking pin 114 in a downward or upward motion.

For example, a lock plate 116 mounted along the locking pin 114 can be urged downwardly by the pivoting lock member 113 to move the locking pin 114 downward, so as to cause or enable the talus and the prosthetic foot attached thereto, to pivot or be moved, e.g. the heel 18A of the foot 18 can be raised and the toe portion 18B pivoted down, as needed to provide a natural locomotion and push-off as the user walks. In response to the sensor 111 sensing a further change in position or angle of the tibial section, e.g. when the user steps with their opposite foot or stops walking, the locking pin 114 can be released from engagement by the locking member 113, allowing a spring 117 to retract the locking pin 114 as the ankle dorsiflexion spring 104 urges the heel of the prosthetic foot downward, toward a substantially flat-lying alignment/orientation. The use of the electromechanical actuator may enable even further increased control and/or precision of the realignment of the talus and foot portion of the prosthetic device, without limiting or restricting such movement to a particular angle or range or angles between the foot and tibial sections.

The use of the orthotic (or brace) and/or prosthetic ankle joint 5 according to the principles of the present disclosure thus can enable the talus of a user to be substantially automatically moved to a new alignment or orientation with respect to the tibial section to accommodate movement of the user along upward or downward slope or terrain. And, as the talus becomes realigned with respect to the tibial section or otherwise moved into an alignment that substantially matches or accommodates the sloped surface along, the lock may be reengaged to potentially secure the position or alignment of the talus with respect to the tibial section. As a further result, the orthotic (or brace) and/or prosthetic ankle joint is enabled to operate in a manner that may substantially mimic the natural movement or swinging operation of the user's ankle joint as the user encounters a sloped surface during ambulation.

Accordingly, embodiments of the disclosure also include a method to operate an orthotic or prosthetic ankle joint or brace for accommodating movement of a user along a slope. An embodiment of a method, for example, may include mounting a joint or brace to an ankle region of a user. The orthotic (or brace) and prosthetic ankle joint includes a tibial section, a talus movably connected to the tibial section, and an adjustment mechanism connected to one or more of the talus or the tibial section and configured to adjust an alignment of the talus with respect to the tibial section. The adjustment mechanism may include a lock as shown and described. The method also may include, when moving the angle of a user's shin forwardly to a selected angle, moving a proximal or engaging end of a lock of the joint or brace into engagement with the teeth of the talus section or ankle portion of the joint or brace, locking the tibial section and talus at the user selected angle when raising a heel of a foot of a user onto which the joint or brace is mounted, and enabling push off as part of the user's normal gait during a walking motion. When the user stops walking, and the user's shin tilts back toward a more natural alignment/orientation with respect to the user's talus, the lock can be disengaged until the talus is realigned in a terminal, standing or rest position, and then reengaged with the talus for supporting the user in either a standing position or as the user takes a step with their opposite foot, thereby to secure the talus and tibial section together for accommodating movement of a user along a slope.

In some embodiments, the adjustment mechanism further includes a pendulum adapted to move in response to the user moving along a slope for locking and unlocking the lock to enable the movement and realignment of the talus with respect to the tibial section and a foot of the ankle joint being coupled to the talus. In other embodiments, the adjustment mechanism further includes an actuator for engaging and disengaging the lock to enable movement of the talus with respect to the tibial section, and the actuator may include one or more of a mechanical actuator or an electromechanical actuator.

The foregoing description generally illustrates and describes various embodiments of the present disclosure. It will, however, be understood by those skilled in the art that various changes and modifications may be made to the above-discussed construction of the present disclosure without departing from the scope of the disclosure as disclosed herein, and that it is intended that all matter contained in the above description or shown in the accompanying drawings shall be interpreted as being illustrative, and not to be taken in a limiting sense. Those skilled in the art will envision other modifications within the scope of the claims appended hereto.

Furthermore, the scope of the present disclosure shall be construed to cover various modifications, combinations, additions, alterations, etc., above and to the above-described embodiments, which shall be considered to be within the scope of the present disclosure. Accordingly, various features and characteristics of the present disclosure as discussed herein may be selectively interchanged and applied to other illustrated and non-illustrated embodiments of the disclosure, and numerous variations, modifications, and additions further may be made thereto without departing from the spirit and scope of the present disclosure as set forth in the appended claims.

What is claimed is:

1. An orthotic or prosthetic ankle joint, the joint comprising:
a tibial section;
a talus section movably connected to the tibial section; and
an adjustment mechanism configured to selectively prevent additional dorsiflexion of the talus section relative to the tibial section to accommodate movement of a user.

2. The joint of claim 1, wherein the adjustment mechanism comprises a lock and a pendulum, the pendulum adapted to move in response to movement of the user such that the lock is locked to prevent additional dorsiflexion of the talus section with respect to the tibial section and unlocks to allow realignment of the talus section with respect to the tibial section.

3. The joint of claim 2, wherein when locked, the lock fixes the talus section with respect to the tibial section.

4. The joint of claim 2, further comprising a foot coupled to the talus section.

5. The joint of claim 2, wherein the talus section includes a series of teeth engagable by the lock such that engagement of the lock with a tooth of the series of teeth prevents additional dorsiflexion of the talus section with respect to the tibial section.

6. The joint of claim 5, wherein the lock is pivotally mounted to the tibial section and comprising an engaging end, the engaging end for engaging the series of teeth to prevent additional dorsiflexion of the talus section with respect to the tibial section.

7. The joint of claim 6, wherein the lock is operably coupled to the pendulum such that the pendulum pivots the engaging end into and out of engagement with the series of teeth.

8. The joint of claim 5, wherein the lock is slidably mounted to the tibial section and comprises an engaging end, the engaging end for engaging the series of teeth to prevent additional dorsiflexion of the talus section with respect to the tibial section.

9. The joint of claim 8, wherein the lock includes a rack, the pendulum operably coupled to a pinion engaged with the rack, the pendulum pivoting the pinion to translate the lock into and out of engagement with the series of teeth.

10. The joint of claim 2, wherein the tibial section is pivotably connected to the talus section by a connector, the adjustment mechanism comprises a pendulum pivotably disposed about the connector, the tibial section comprises a series of teeth, the lock operably coupled to the pendulum such that the pendulum moves the lock into and out of engagement with the series of teeth to prevent additional dorsiflexion of the talus section with respect to the tibial section.

11. The joint of claim 10, wherein the talus section includes a projecting portion that includes the series of teeth, the projecting portion defining a cavity, the lock disposed within the cavity of the projecting portion.

12. The joint of claim 1, wherein the adjustment mechanism further comprises a lock connected to one or more of the talus section or tibial section, and an actuator for engaging the lock to prevent additional dorsiflexion of the talus section with respect to the tibial section and disengaging the lock to enable movement of the talus section with respect to the tibial section.

13. (canceled)

14. The joint of claim 1, wherein the adjustment mechanism comprises a position sensor and an electromechanical actuator, the position sensor configured to sense a change in orientation or direction of movement of the talus with respect to the tibial section, the electromechanical actuator configured to selectively allow movement of the talus section with respect to the tibial section in response to the orientation or the direction of movement of the talus section with respect to the tibial section.

15. The joint of claim 14, wherein the adjustment mechanism comprises a locking pin, the locking pin translatable between a first position in which movement of the talus section with respect to the tibial section is allowed and a second position in which additional dorsiflexion of the talus section with respect to the tibial section prevented, the electromechanical actuator operably coupled to the locking pin to translate the locking pin between the first position and the second position.

16. (canceled)

17. The joint of claim 1, further comprising a biasing member connected to the talus section and the tibial section, the biasing member configured to cause or aid dorsiflexion of the talus section with respect to the tibial section.

18. A method to operate an orthotic or prosthetic ankle joint for accommodating movement of a user along a slope, the method comprising:
mounting a joint to an ankle region of a user, the joint including a tibial section, a talus section movably connected to the tibial section, and an adjustment mechanism configured to adjust an alignment of the talus section with respect to the tibial section, the adjustment mechanism including a lock;
moving an engaging end of the lock into engagement with teeth of the talus section when an angle of a user's shin moves forwardly to a user selected angle;
locking the tibial section and talus section at the user selected angle;
enabling push off as part of the user's normal gait during a walking motion when raising a heel of a foot of a user onto which the joint is mounted; and
reengaging the talus section with the lock to secure the talus section and the tibial section together for accommodating movement of a user along a slope.

19. The method of claim 18, further comprising locking and unlocking the lock with a pendulum in response to the user moving along a slope to enable movement and realignment of the talus section with respect to the tibial section.

20. The method of claim 19, further comprising a foot coupled to the talus section.

21. The method of claim 18, further comprising engaging and disengaging the lock with an actuator to enable movement of the talus section with respect to the tibial section.

22. The method of claim 21, wherein engaging and disengaging the lock includes the actuator comprising a mechanical actuator or an electromechanical actuator.
